(19) United States
(12) Patent Application Publication
(10) Pub. No.: US 2015/0335356 A1
(43) Pub. Date: Nov. 26, 2015

(54) Skin Treatment Apparatus

(71) Applicants: Rodge Brooks, Plano, TX (US); Andrea Morley Brooks, Plano, TX (US)

(72) Inventors: Rodge Brooks, Plano, TX (US); Andrea Morley Brooks, Plano, TX (US)

(21) Appl. No.: 14/585,652

(22) Filed: Dec. 30, 2014

Related U.S. Application Data
(60) Provisional application No. 62/041,680, filed on Aug. 26, 2014.
(60) Provisional application No. 62/001,948, filed on May 22, 2014.

(51) Int. Cl. A61B 17/54

(52) U.S. Cl. CPC A61B 17/54

(57) ABSTRACT

In various implementations, a skin treatment device may include an abrasive member. The abrasive member may be perforated. The skin treatment device may include flange(s) that at least partially retain the abrasive sheet in a recess of the body of the skin treatment device. In various implementations, the skin treatment device may be utilized to remove calluses.

### PRIORITY

The present application claims priority to provisional application 62/041,680 filed Aug. 26, 2014 and provisional application 62/001,948 filed May 22, 2014, the entire of both of which are incorporated herein by reference.

### BACKGROUND OF THE INVENTION

1. Technical Field

The present invention relates to skin treatment devices, and more particularly to a device for skin treatment.

2. Description of Related Art

Currently, callus removal includes abrasion of the skin with files and other devices that often damage the skin in addition to removing calluses from areas, such as feet. For example, people often use metal cutters (shavers and/or rasps) with sharp surfaces that can damage and/or remove too much of the skin. A user may inadvertently shave off too much skin and damage skin and/or draw blood. Pumice stones and files (e.g., diamond and/or amen) are also commonly used.

### SUMMARY

In various implementations, a skin treatment device may be utilized to remove at least a portion of the skin from an area. For example, the skin treatment device may be used to remove calluses, corns, and/or a portion of layers) of skin. The skin treatment device may be rubbed against a portion of a body, such as a foot, to remove calluses, corns, and/or a portion of layers) of skin.

In some implementations, the skin treatment device may include a body with a perforated abrasive sheet. The skin treatment device may include one or more retaining members to couple the abrasive sheet to the body of the skin treatment device. For example, the retaining members may include flanges that retain the abrasive member in a recess of the body and/or protrusions adapted to couple with the abrasive member. During use, the openings of the perforated abrasive member may inhibit fouling of the device (e.g., clogging openings and/or at least partially coating abrasives of the abrasive member such that abrasion is limited).

The details of one or more implementations are set forth in the accompanying drawings and the description below. Other features, objects, and advantages of the implementations will be apparent from the description and drawings.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1A illustrates a perspective view of an implementation of an example skin treatment device;

FIG. 1B illustrates a perspective view of an implementation of the example skin treatment device illustrated in FIG. 1A;

FIG. 2A illustrates a side view of an implementation of an example skin treatment device;

FIG. 2B illustrates a perspective view of an implementation of the example skin treatment device illustrated in FIG. 2A;

FIG. 3A illustrates a perspective view of an implementation of an example first member of a body of a skin treatment device;

FIG. 3B illustrates a perspective view of an implementation of the example first member illustrated in FIG. 3A;

FIG. 4 illustrates a perspective view of an implementation of an example second member of a body of a skin treatment device;

FIG. 5 illustrates an implementation of an example abrasive material;

FIG. 6A illustrates a side view of an implementation of an example skin treatment device;

FIG. 6B illustrates a perspective view of an implementation of the example skin treatment device illustrated in FIG. 6A;

FIG. 6C illustrates a bottom view of an implementation of the example skin treatment device illustrated in FIG. 6A;

FIG. 6D illustrates an implementation of an example additional retaining member of a skin treatment device;

FIG. 6E illustrates an implementation of an example additional retaining member of a skin treatment device;

FIG. 7 illustrates an implementation of a portion of an example skin treatment device;

FIG. 8A illustrates a perspective view of an implementation of an example body of a skin treatment device;

FIG. 8B illustrates a perspective view of an implementation of the example body illustrated in FIG. 8A;

FIG. 8C illustrates a perspective view of an implementation of a portion of the example body illustrated in FIG. 8A.

FIG. 9A illustrates a perspective view of an implementation of an example body of a skin treatment device;

FIG. 9B is a side view of the example body shown in FIG. 9A;

FIG. 10A illustrates a perspective view of an implementation of an example body of a skin treatment device;

FIG. 10B is a side view of the example body shown in FIG. 10A;

Like reference symbols in the various drawings indicate like elements.

### DETAILED DESCRIPTION OF THE EMBODIMENTS

Skin treatment using the skin treatment device may include removing a portion of the skin, such as removing a portion of one or more layers of skin. For example, calluses may form on feet or other areas of the skin. Portions of the skin may thicken and/or discolor. Thus, a user may remove at least a portion of layer(s) of the skin; e.g., one or more layers of cells and/or at least a portion of the epidermis, as a skin treatment using the skin treatment device. A user may use the skin treatment device, for example, on his/her own skin and/or on another user's skin.

The skin treatment device may be utilized wet and/or dry. For example, when utilizing the skin treatment device in a wet application, liquids, such as water, moisturizer, enzyme peels, etc., may be applied to the skin treatment device, skin, and/or portions thereof. When utilizing the skin treatment in a dry application, the skin treatment device may be rubbed against skin without application of liquids.

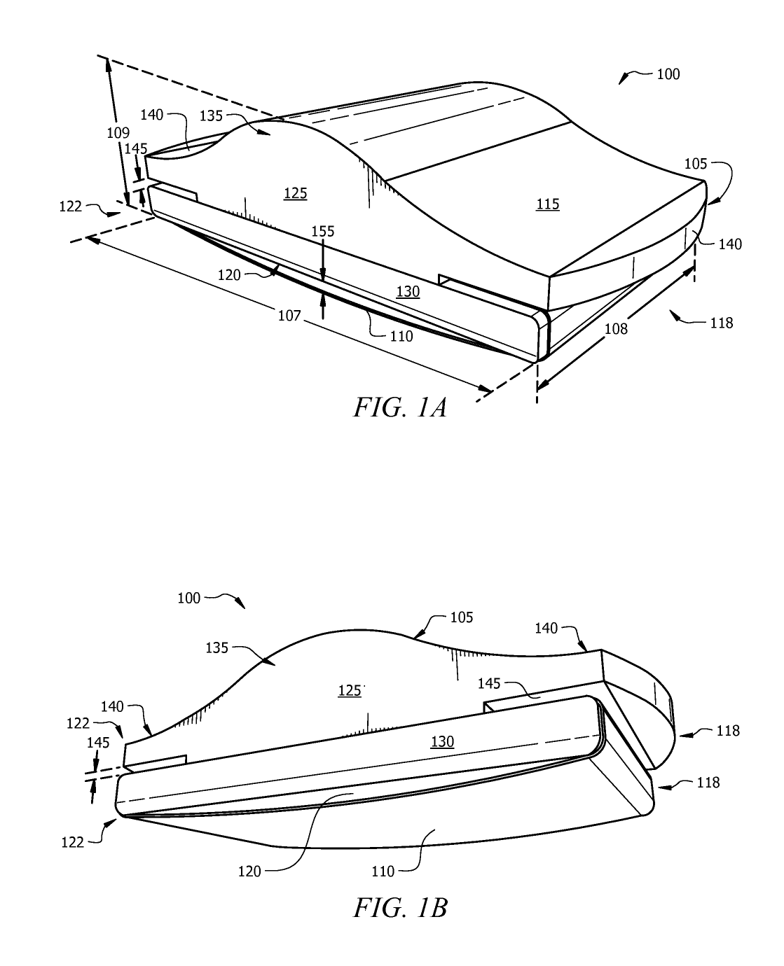

In various implementations, the skin treatment device may include a body and an abrasive member. In some implementations, the abrasive member may include a perforated abrasive member. The perforated abrasive member may inhibit fouling; e.g., coating of abrasive with skin, dirt, and/or debris during use during use and/or facilitate 5 cleaning of the skin treatment device. FIG. 1A illustrates a perspective view of an implementation of an example skin treatment device 100. FIG. 1B illustrates a perspective view of an implementation of the example skin treatment device 100, illustrated in FIG. 1A. The skin treatment device 100 may include a body 105 and an abrasive member 110. The abrasive member 110 may be coupled to; e.g., attached to, affixed to, and/or retained by the body 105.

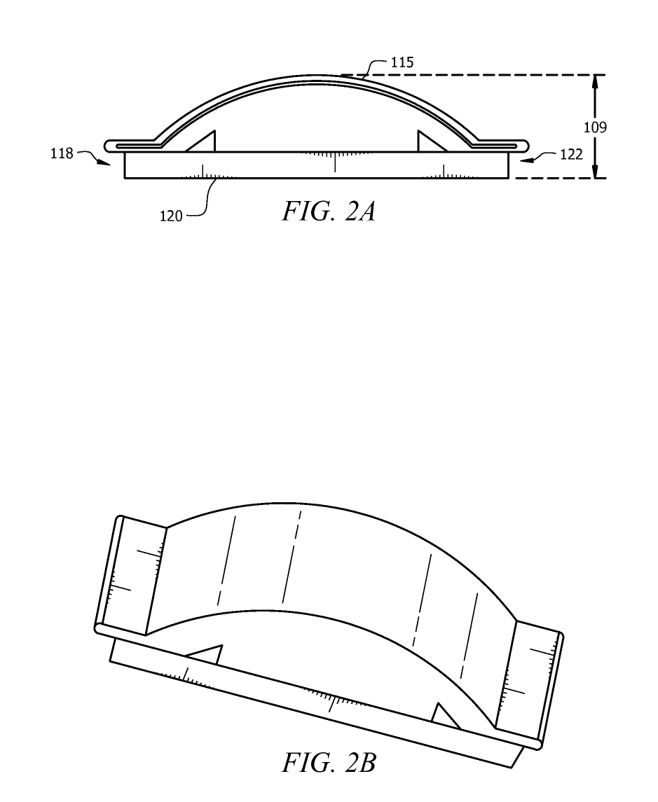

FIG. 2A illustrates a side view of an implementation of a body 105 of the skin treatment device. FIG. 2B illustrates an implementation of a perspective view of an implementation of the example body 105, illustrated in FIG. 2A. As illustrated, the body 105 may have a length 107, a width 108, and a height 109. The body may have any appropriate size. In some implementations, the size of the body 105 may be selected such that the body and/or portions thereof may fit in a hand of a user; e.g., smallest, median, average, and/or largest hand size of a population. In some implementations, the length may be approximately 100 mm to approximately 150 mm; the width 108 of the body 105 may be approximately 25 mm to approximately 80 mm; and/or the height may be approximately 20 to approximately 50 mm.

The body 105 may include a top surface 115 and a bottom surface 120. The body 105 may include a first side 118 and a second side 122. The abrasive member 110 may at least partially cover the bottom surface 120 of the body 105. A first portion of the abrasive member 100 may wrap around at least a part of the first side 118 of the body to allow a first end of the abrasive member to be at least partially disposed in a first recess 145 of the body 105. A recess 145 is a void which can receive at least a portion of an abrasive member 100. In one embodiment, the recess 145 is a gap between the top surface 115 and a bottom surface 120. A second portion of the abrasive member 110 may wrap around at least a part of the second side 122 of the body 105 to allow a second end of the abrasive member, disposed opposite the first end, to be at least partially disposed in a second recess 145 of the body 105. At least a portion of the abrasive member 110 between the first portion and the second portion of the abrasive member may be disposed proximate the bottom surface 120 of the body 105.

The body 105 may include any appropriate material. For example, the body 105 may include rigid plastics, nonrigid plastics; e.g., elastomers, rubber, metal (e.g., aluminum), etc. The body 105 may include more than one material, in some implementations. For example, the body may include a rigid portion and a flexible portion. The rigid portion may include rigid plastics; e.g., thermosest plastic and/or thermoplastic, such as acrylonitrile butadiene styrene (ABS) plastics, and the flexible portion may include resins including elastomer and/or rubber.

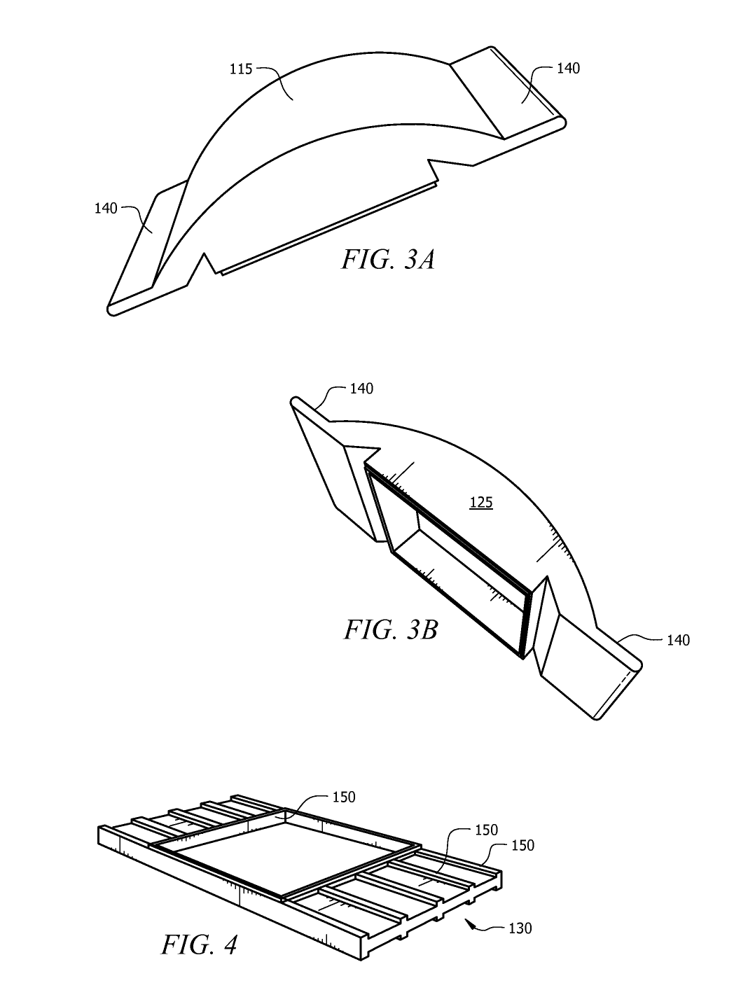

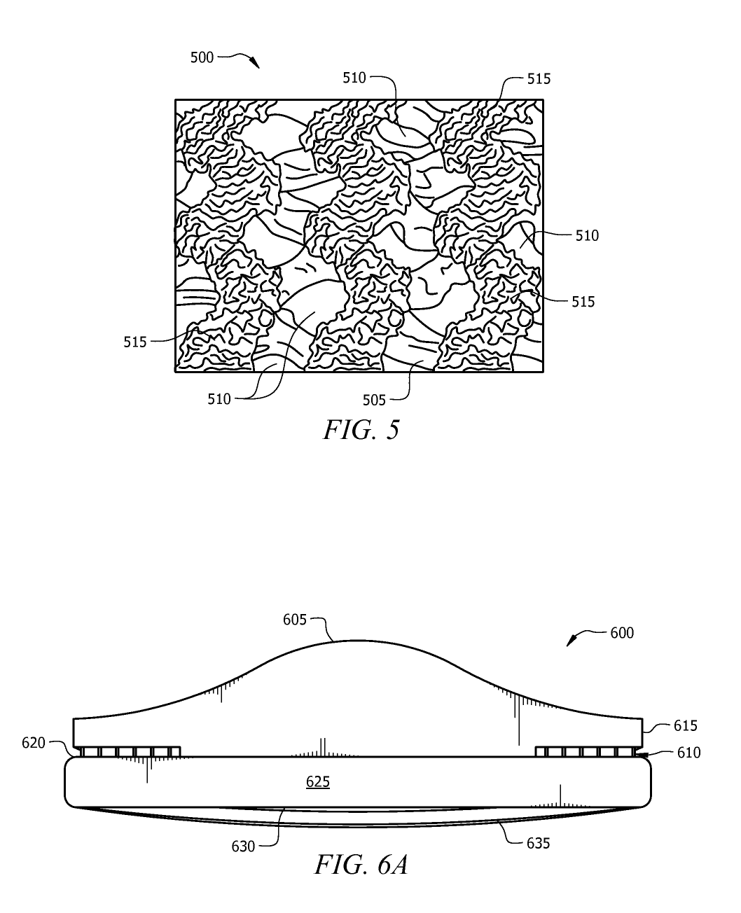

The body 105 may include more than one member coupled together. FIG. 3A illustrates a top view of an implementation of an example first member 125. FIG. 3B illustrates a bottom view of an implementation of the example first member 125, illustrated in FIG. 3A. FIG. 4 illustrates a bottom view of an implementation of an example second member 130. As illustrated, the body 105 may include a first member 125 and a second member 130.

The first member 125 may form at least a portion of top surface 115 of the body 105, in some implementations. The first member 125 may include a handle 135 and one or more flanges 140. The handle may be formed a contoured region of the first member, in some implementations. The handle may include recesses, protrusions, grips, etc. to aid retention of the device by a user during use. The handle may include an oval-molded grip to facilitate use of the device; e.g., to make retaining the skin treatment device in a hand while rubbing skin. For example, for a user holding the skin treatment device 100 in a hand, the grips may facilitate retaining the skin treatment device in the hand.

The body 105 may include one or more flanges 140. As illustrated, the body 105 may include two opposing flanges 140. The flanges 140 may be flexible. For example, a flange 140 may include a flexible material to allow deformation of the flange 140. A flange 140, in on embodiment, is a flexible member which can be manipulated to couple an abrasive member. The first member 125 may include a flexible portion of the body 105. In some implementations, the flexible portion may be an oval-molding applied to the first member and/or body. The first member 125 may include a rigid portion, in some implementations. The flexible portion may include the flange(s). By allowing the flange(s) of the body to be flexible, a flange may be retracted from an initial position; e.g., proximate a top surface of the second member, to one or more other positions away from the top surface of the second member. When the flange is released, the flange may return to approximately the initial position. When the flange is in the initial position or proximate the initial position, the flange may exert pressure on abrasive member(s) 110 in the recess 145 of the body to retain the abrasive member(s) 110 in the recess. Thus, the abrasive member is at least partially disposed in the recess 145.

The second member 130 may be coupled; e.g., snapped into, affixed, bonded, etc., to the first member 125. In one embodiment the first and second members are releasably coupled. In some implementations, the first member 125 and the second member 130 may be a single unit. For example, the first member 125 and the second member 130 may be injection molded into any appropriate shape. In some implementations, a recess 145 may be disposed between a flange 140 of the first member 125 and a top surface of the second member 130.

FIG. 4 illustrates a bottom view of an implementation of an example second member 130. The second member may include a rigid portion. The rigid portion may resist deformation during use of the skin treatment device. For example, when a user rubs skin with the skin treatment device, the second member may resist deformation due to the force applied by the user.

The second member 130 may include one or more hollow portions, one or more solid portions, and/or one or more supporting members. The supporting members may increase the rigidity of the second member 130 and/or the body 105; e.g., when compared to a second member without supporting members, such as a hollow portion without supporting members.

The second member 130 may include at least a portion of the bottom surface in some implementations. The second member 130 may include ridges 150; e.g., protrusions, proximate the bottom surface of the body 105. As illustrated, the ridges 150 may form a pattern or the ridges may be irregularly arranged on the second member 130. The ridges may add strength or rigidity to the body of the skin treatment device. By utilizing ridges rather than increasing a height of the body, the weight increase associated with adding the additional strength; e.g., via the ridges, may be minimized.

In some implementations, the ridges may create a separation 155 between a portion of the bottom surface of the second member and the abrasive member coupled to the body. The separation 155 may allow skin, dirt, and/or debris removed from skin to which treatment is applied, fall between openings in the abrasive member and/or through the separation.

In some implementations, the abrasive member may be coupled to the body of the skin treatment device such that a separation 155 is created, rather than or in addition to creating a separation using the ridge(s).

In various implementations, the skin treatment device 100 may include an abrasive member 110 coupled to the body of the skin treatment device. The abrasive member 110 may be removably coupled to second member 130 to the body 105 of the skin treatment device 100. Thus, an abrasive member may be replaced with another abrasive member; e.g., similar or different type, and/or the abrasive member may be removed to facilitate cleaning; e.g., of the abrasive member and/or the body.

To couple the abrasive member to the body, an end of the abrasive member 110 may be disposed in the recess 145 between a flange 140 and a top surface of the second member 130. The flange 140 may at least partially retain the end of the abrasive member at least partially in the recess. At least a portion of the abrasive member may be flexible. Thus, a portion of the abrasive member may be disposed proximate a bottom surface of the body and portions proximate each end of the abrasive member may wrap around an end of the second member such that an end of the abrasive member may be disposed in the recess of the body.

The abrasive material may include any appropriate abrasive material. For example, the abrasive material may include a perforated abrasive material that is capable of rubbing or scraping skin without substantially damaging the skin. The abrasive material may include an abrasive sheet in some implementations. The abrasive sheet may have a length, a width, and a height. The height of the abrasive sheet may be less than the length and the width.

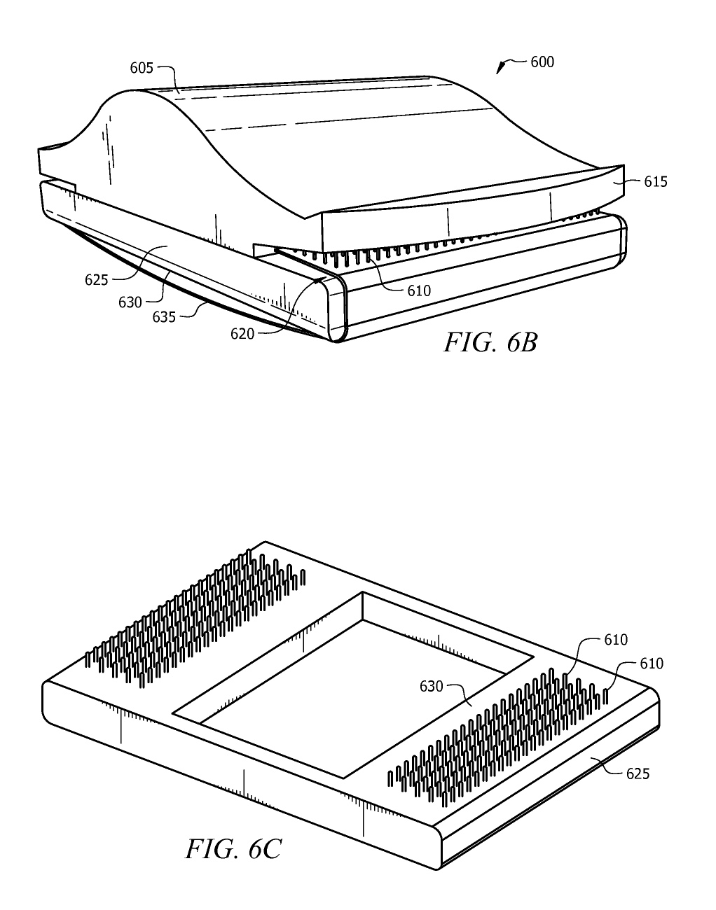

Any appropriate abrasive member may be utilized. FIG. 5 illustrates an implementation of a portion 500 of an example abrasive member. As illustrated, the abrasive member may be a perforated abrasive sheet. For example, the abrasive member may include Abranet and/or Abranet Soft, commercially available from Mirka (Twinsburg, Ohio). The perforated abrasive sheet may include an abrasive mesh. The perforated abrasive sheet may include a support 505 and one or more openings 510 disposed in the support. For example, the support of the abrasive material may include a mesh, screen, a lattice formation, etc. The openings 510 disposed in the support may include any appropriate shape. In some implementations, the support may include a fabric mesh and/or plastic mesh. For example, fabric and/or plastic mesh may be coated and/or impregnated with an abrasive. In some implementations, the fabric and/or plastic mesh may be abrasive.

An abrasive 515 may be coupled to the support 505. Any appropriate abrasive for skin treatment may be utilized. For example, an abrasive such as silicone carbide or aluminum oxide may be coupled; e.g., bonded, affixed, and/or coated, to the support of the abrasive material. In some implementations, the perforated abrasive sheet may include mesh coated with an abrasive material, such as silicone carbide.

The abrasive selected may be based on the amount of abrasion sought by a user during a skin treatment. For example, a user may first utilize a first coarse abrasive material with the skin treatment device and then a more fine abrasive material with the skin treatment device. In some implementations, the abrasive material selected may be based on the area in which the skin treatment device may be utilized. For example, a courser abrasive material may be utilized when a user applies the skin treatment device to a foot and a finer abrasive material may be utilized when a user applies the skin treatment device to a hand or an elbow.

Although FIGS. 1A-5 illustrates implementations of a skin treatment device, in some implementations, the skin treatment device may include one or more of these features and/or other features. For example, the second member may not include ridges. In some implementations, the ridges may not form a pattern. One or more of the ridges of the second member may be formed by recesses in the bottom surface of the second member. The abrasive member may include more than one type of abrasive. The abrasive member may be coupled to the body of the skin treatment device using more than two recesses. The handle may include a contoured portion of the handle adapted to fit in a hand of the user. In some implementations, the skin treatment device may include a flange that extends across more than one side of the body. The skin treatment device may include more than two flanges, in some implementations. The skin treatment device may not include a separation between the abrasive member and a portion of the bottom surface of the second member of the body.

In some implementations, the first member may include an overmolding. For example, the first member may include at least a first part and a second part. The first part of the first member may couple with the second member. The first part may include a rigid substrate, such as a rigid plastic. The second part may at least partially form the handle top surface, and/or flange(s). The second part may include an overmolded elastomer; e.g., an injection molded thermoplastic elastomer. The first part and the second part may be coupled; e.g., via the injection molding that forms each piece, in some implementations. During use, a user may hold the skin treatment device, for example, by gripping the handle. For example, fingers of a user's hand may wrap at least partially around a handle of the body of the skin treatment device. The user may then rub; e.g., contact the abrasive member with a location on the skin and move the abrasive member and/or the portion of the body on which the location resides in a back and forth motion, a circular motion, etc. As the skin treatment device is applied to the skin, at least a portion of at least one layer of skin; e.g., cells and/or layers, may be removed; e.g., detached, scraped, abraded etc., from the skin. At least a portion of the removed skin may fall through the openings of the abrasive member and/or the separation between the abrasive member and the body of the skin treatment device. By allowing at least a portion of the removed skin to fall through the openings of the abrasive member and/or the separation, fouling of the skin treatment device may be inhibited. In addition, cleaning of the skin treatment device may be facilitated and/or required less often due to lack of fouling.

In some implementations, the abrasive sheet may be removed and/or replaced. A skin treatment device may include a body with at least two elastically deformable flanges. To couple an abrasive member to the body of the skin treatment device, a user may move at least a portion of a flange, from an initial position, away from the second member by deforming the flange. The flange may be elastically deformable. Thus, the flange may be at least partially bent to move an end of the flange away from a top surface of the second member and/or the recess of the body. By deforming the flange, the size of the recess may be increased. The abrasive material may be a sheet with at least two ends. At least a portion of the sheet may be disposed proximate the bottom surface of the body. At least a portion of the abrasive material may be at least partially wrapped around a first end of the body. At least a portion of a first end of the abrasive material may be inserted into the recess by the user. The flange may then be released by the user such that the flange returns to approximately the initial position; e.g., since the flange is elastically deformable. When the flange is in the initial position, the flange may allow contact between at least a portion of the abrasive material and the flange and/or the second member. Thus, the abrasive material may be at least partially retained by the flange. The second end of the abrasive material may be similarly disposed in an opposing recess by the second opposing flange. The second flange may be deformed to move the second flange away from the top surface of the second member and increase the size of the recess. A portion of the abrasive material opposing the first end of the abrasive material may be wrapped at least partially around the second end of the second member. The second end of the abrasive material may then be positioned at least partially in the second recess. The second flange may then be released to retain the second end of the abrasive material or at least a portion there of to be retained in the second recess; e.g., the second end of the abrasive material may be at least partially retained by contact with the second flange and the top surface of the second member. Thus, both ends of the abrasive material may be retained in the recesses of the body. The abrasive material may be disposed proximate the bottom surface of the body such that a separation may reside between the abrasive material and the bottom surface of the second member and/or body. During use, the existence of the separation may allow skin, dirt, and/or debris that is removed by the skin treatment device to fall between openings of the abrasive material and/or the body of the skin treatment device to inhibit fouling of the abrasive material.

The abrasive material may be removed from a body of the skin treatment device, in various implementations, to replace the abrasive material with another abrasive material; e.g., same or different type of abrasive material, and/or to remove the abrasive material; e.g., to store the skin treatment device, to clean the skin treatment device, etc. To remove the abrasive member, each flange may be deformed to move an end of each flange away from a top surface of the second member and to increase the recess size.

The abrasive member may then be removed from the recess. In some implementations, a force may be applied to the abrasive member; e.g., proximate an end of the abrasive member, to release the abrasive member from the recess. Once the abrasive member is uncoupled from the body, the skin treatment device or portions thereof may be cleaned, the abrasive member may be replaced, etc. If the abrasive member is to be replaced; e.g., after cleaning and/or with a different abrasive member, then the flanges of the body of the skin treatment device may be deformed to increase the space of the recess and allow insertion of ends of the abrasive member at least partially in the recess.

In some implementations, a kit may be provided that includes the body of the skin treatment device and more than one abrasive material. For example, the abrasive materials in the kit may include replacement abrasive sheets, abrasive sheets with different abrasion properties; e.g., course, fine, buffer, etc., different types of abrasive sheets such as perforated and/or non-perforated, coated mesh, abrasive mesh, etc.

A user may select an abrasive material for attachment to the body of the skin treatment device. In some implementations, a skin treatment device may be coupled to a default abrasive material in the kit. To couple an abrasive material to the body of the skin treatment device, each flange may be deformed to increase a recess to receive at least a portion of an end of the abrasive material and then releasing the flange to retain the portion of the abrasive material in one or more recesses. In some implementations, the user may decide to remove the abrasive material coupled to the skin treatment device, for example, to clean the body of the skin treatment device and/or replace the abrasive material. To remove an abrasive material from the body of the skin treatment device, each flange that retains a portion of the abrasive material may be deformed to increase the recess in which a portion of the abrasive material resides to allow the abrasive material to be released and/or removed. In some implementations, the user may select another abrasive material from the kit to couple to the skin treatment device; e.g., after an abrasive material has been removed from the device. The user may select the abrasive member, e.g., to replace a worn abrasive material and/or to select a different type of abrasive member.

The user may then deform; e.g., retract, each flange to insert an end of the abrasive member in the recesses of the body. The user may then insert an end of the abrasive member in the recesses and then release the flange such that the abrasive material is retained in the recesses of the body.

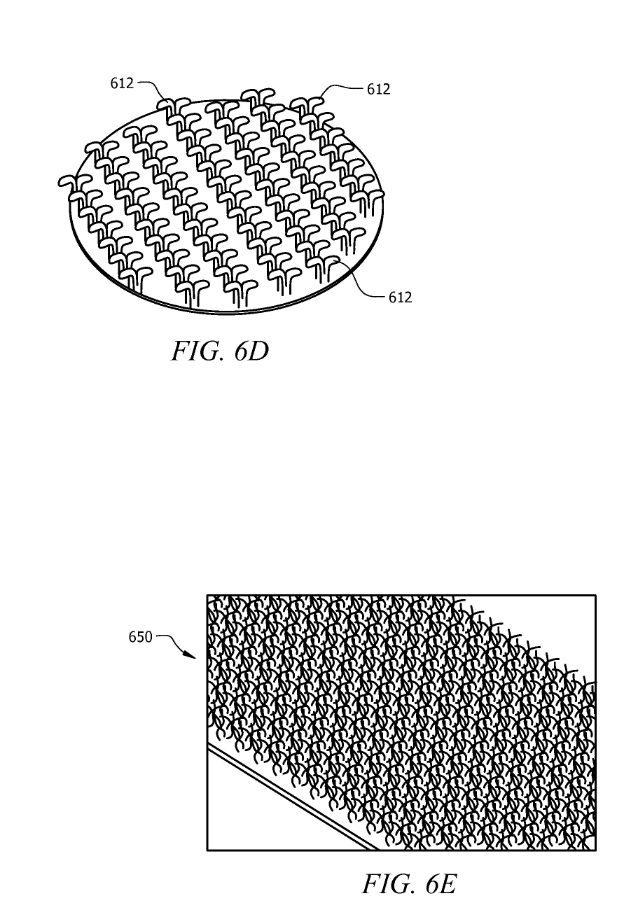

In various implementations, the flanges, recesses, and or surfaces of the second member may be retaining members for the body of the skin treatment device and at least partially couple the abrasive member with the body. In some implementations, the skin treatment device may include one or more additional retaining members. FIG. 6A illustrates a side view of an implementation of an example skin treatment device 600.

FIG. 6B illustrates a perspective view of an implementation of an example skin treatment device 600. As illustrated, the body 605 of the skin treatment device 600 may include protrusions 610; e.g., sharp and/or blunt spikes, hooks, etc., in addition to and/or instead of flanges 615. The protrusions 610 may be disposed on the top surface 620, the bottom surface 625 of the second member 630, the body 605, and/or portions thereof.

FIG. 6C illustrates a bottom view of an implementation of an example second member 630. As illustrated, the protrusions 610 may be disposed on a bottom surface 630 of the second member 625. The protrusions 610 may be disposed proximate two of the opposing ends of the body, in some implementations with at least a portion of the abrasive member 635 protrusions may puncture and at least partially pass at least through the abrasive member. The protrusions 610 may couple with at least a portion of the abrasive member 635. As illustrated in FIG. 6B, the protrusions may puncture and at least partially pass at least through the abrasive member.

FIG. 6D illustrates an implementation of an example protrusion that includes a hook 612. A protrusion that includes a hook may couple with an opening in a perforated abrasive member. A hook may couple with loops on a surface of the abrasive member. FIG. 6E illustrates an implementation of loops 650 that may be utilized on a surface of the abrasive member. For example, an abrasive member may include loops on a surface to couple with the hooks of the skin treatment device. In some implementations, the abrasive member may include hooks; e.g., on an opposing side to the abrasive side and/or on the side to be coupled proximate the bottom surface of the second member, and a surface of the skin treatment device may include loops to couple with the hooks of the abrasive member.

In some implementations, the abrasive member may include additional retaining members. The abrasive member may include an adhesive; e.g., removable adhesive and/or adhesive adapted to secure objects for a predetermined time period, on a portion of the abrasive member to couple with the body of the skin treatment device. For example, the abrasive member may include adhesive strips proximate end(s) of the abrasive member. When the ends are wrapped around the ends of the second member, the adhesive strip may be utilized to couple the end to a top surface and/or edge of the second member. In some implementations, the adhesive strips may allow the abrasive member to be coupled to at least a portion of the bottom surface of body; e.g., second member. In some implementations, the additional retaining member may be coupled to the body of the skin treatment device. For example, the additional retaining member may include a retaining layer. The retaining layer may include a first surface with the additional retention member; e.g., hooks/loops, and a second opposing surface. The second opposing member may allow coupling to the body; e.g., the bottom surface of the second member of the body. The retaining layer may be coupled; e.g., fastened, affixed, bonded, glued, and/or otherwise coupled) to at least a portion of a bottom surface of the body via the second surface. For example, the retention layer may include Velcro tape with hooks and/or loops on a first surface and an adhesive; e.g., glue, on the second surface. The retention layer may be bonded, heat set, and/or otherwise coupled to the body. In some implementations, the retention layer may be adapted to remain coupled to the body when an abrasive member is uncoupled from the body.

In some implementations, the skin treatment device may utilize one or more of the additional retaining members in addition to and/or instead of one or more of the flanges of the body. For example, the body of the skin treatment device may not include flanges, but may include hooks adapted to couple with loops on an abrasive member.

In some implementations, the additional retention member may couple the abrasive member and the body rather than the flange(s) of the body. In some implementations, the body may not include flanges and/or recesses to couple the body and the abrasive member. For example, the body and the abrasive member may be coupled via adhesive, hooks/loops, protrusions, fasteners, etc; and, the abrasive member may not wrap around at least a portion of a side of the body to be received in the recess of the body and retained by the flange, in some implementations. The flanges may facilitate gripping of the skin treatment device by the user, in some implementations.

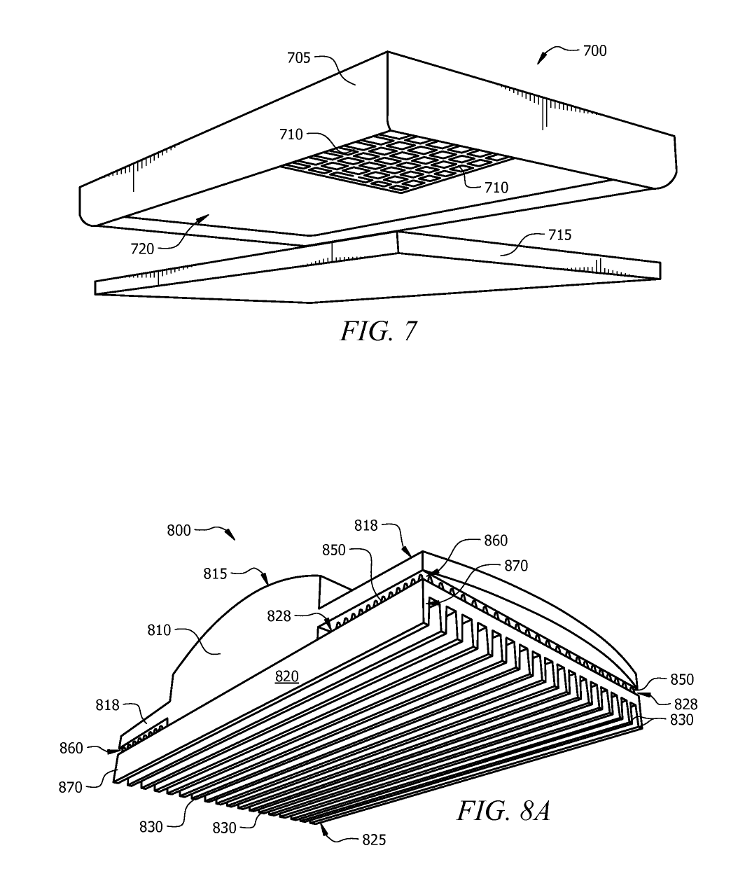

In some implementations, the body of the skin treatment device may be adapted to be lightweight. FIG. 7 illustrates a portion 700 of the second member of the body of the skin treatment device. For example, the second member and/or the body may include a frame 705 with one or more openings 710. The frame may provide structural strength; e.g., rigidity, and the openings may decrease the weight of the frame; e.g., when compared with a solid frame, without decreasing the strength of the frame less than a predetermined strength. Reducing the weight of the frame may be cost effective, since less material may be included in the skin treatment device body. In some implementations, the body may include a foam member 715; e.g., open cell foam and/or closed cell foam, such as approximately 1 mm to approximately 3 mm closed cell foam and/or approximately 2 mm to approximately 3 mm closed cell foam. The foam member may be disposed at least partially in the frame. As illustrated, the frame 705 may include a recess 720 in which the foam may be at least partially disposed. The recess 720 may be disposed between walls, such as rounded walls, of the frame 705. The dimensions of the walls may be selected such that use of the device may not deform the walls of the frame. The walls may have similar thicknesses in some implementations. A bottom surface of the foam may be approximately planar with the plane in which the bottom surface of the frame, in some implementations.

In some implementations, the foam may form at least a portion of the bottom surface of the body. For example, the foam may be dispose proximate at least a portion of the abrasive member. During use, as a user applies; e.g., rubs, the abrasive member on a portion of a body; e.g., a foot, a part of the first side of the abrasive member may contact the portion of the body and a part of the second opposing side of the abrasive member may contact the foam. The foam may provide a padded surface to increase user satisfaction of the device during use.

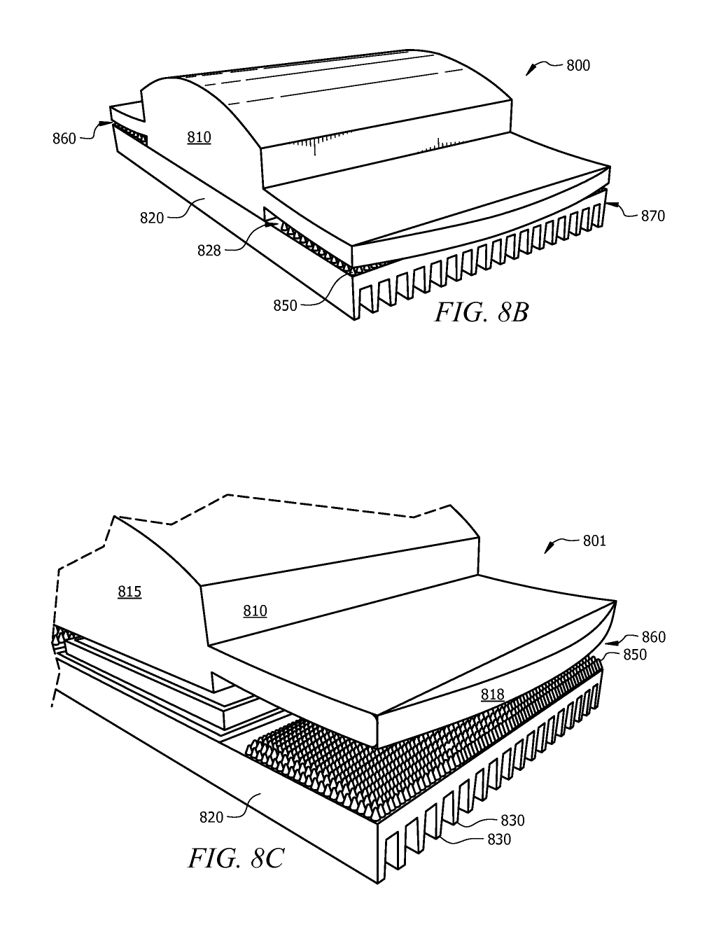

In some implementations, the body of the skin treatment device may include ridges proximate a bottom surface of the body. FIG. 8A illustrates a bottom view of an implementation of the skin treatment device 800. FIG. 8B illustrates a side view of an implementation of the example skin treatment device 800 illustrated in FIG. 8A. FIG. 8C illustrates a portion 801 of an implementation of the example skin treatment device illustrated in FIG. 8A.

As illustrated, the skin treatment device 800 includes a body 805. The body 805 may include a first member 810 and a second member 820. The first member 810 and the second member 820 may be coupled; e.g., bonded, affixed, clipped, etc. In some implementations, the first member 810 and the second member 820 may be molded together; e.g., injection molded together to form a single unit.

The first member 810 of the body 805 may include a handle 815 and one or more flanges 818. In some implementations, the handle 815 may include a protrusion extending from the first member 810. The protrusion may facilitate gripping the skin treatment device by providing an area to hold for the user. The flange(s) of the first member 810 may be flexible. A flange 818 may allow elastic deformation such that when a flange is bent or otherwise deformed from an initial position, when the flange is released, the flange may return to approximately the initial position.

The second member 820 may include ridge(s) 830 proximate the bottom surface 825 of the second member. The ridges may be disposed across the bottom surface 825 of the second member and/or portions thereof. The ridges may include a rigid portion. The ridges may provide structural support for the device during use, may inhibit fouling; e.g., by creating a gap between portions of the second member and the abrasive member through which portions of the skin may fall through, may decrease the weight of the body of the skin treatment device, and/or may increase user satisfaction; e.g., by providing a massaging feel during use of the skin treatment device. The ridges may be formed in a regular pattern, as illustrated, or in other irregular or regular patterns. During use, the ridges may contact the abrasive member and transfer the force that a user applies to the skin care device to the abrasive member such that at least a portion of a skin, receiving the skin treatment, may be removed.

The skin treatment device 800 may include an additional retaining member, such as protrusions 850. The protrusions 850 may be disposed proximate a top surface 828 of the second member 820. The protrusions 850 may be cone shaped, in some implementations. A plurality of protrusions may be disposed on at least a portion of the top surface of the second member. In some implementations, the protrusions may be disposed on the flange in addition to and/or instead of on at least a portion of the top surface of the second member.

When an abrasive member, such as a perforated abrasive sheet, is coupled to the body of the skin treatment device, at least two opposing ends of the abrasive member may be disposed at least partially in the recess 860 of the body. The recess 860 may be formed between the flange 818 and the second member 820. During use, the abrasive member is disposed proximate the bottom surface 825 of the second member. Thus, in some implementations, an abrasive member larger that the bottom surface 825 of the second member may be utilized. One or more ends of the abrasive member may wrap at least partially around an end 870 of the second member and disposed at least partially in the recess. The flange may be retracted; e.g., bent away from the top surface of the second member, to facilitate insertion of an end of the abrasive member and be released to at least partially retain a portion of the abrasive member in the recess. Protrusions 850 may be disposed in the recess to couple with the abrasive member. For example, the protrusions 850 may puncture, grasp, or otherwise retain the abrasive member.

In some implementations, the skin treatment device may include a mesh abrasive member coupled to a body. During use, the user may apply the skin treatment device to a portion of a body; e.g., user's body and/or another user's body, such that the mesh abrasive member contacts the portion of the body. The skin treatment device and/or the body may move such that the mesh abrasive member abrades at least a part of the portion of the body. For example, when a user applies the skin treatment device to a heel, the user may rub the bottom of the skin treatment device against the heel. Thus, at least a portion of the mesh abrasive member may contact and abrade the skin of the heel such that a part of the skin may be removed. The skin may fall off the heel and/or fall through holes in the perforated abrasive member. Thus, fouling of the abrasive member may be inhibited by the openings in the mesh abrasive member.

The use of a mesh abrasive member may increase user satisfaction, in some implementations. A fabric mesh may be softer and/or have less risk associated with over use; e.g., removing too many layers of skin, creating lacerations in the skin, etc., against a user's skin, when compared with metal cutters, metal files, and/or metal graters.

The mesh abrasive member may be flexible. The flexibility of the mesh abrasive member may allow the abrasive member to follow the contours of an area to which the skin treatment device is being applied. For example, the mesh abrasive member may bend to contact more area of a heel; e.g., when compared to a metal or plastic plate such as a metal grater, metal file, plastic grater, metal pumice, etc.

In some implementations, the body may include ridges proximate a bottom surface of the second member of the body. The ridges may extend from the bottom surface of the body or portions thereof. The ridges may create a separation between a surface of at least a portion of the abrasive member and the bottom surface of the body. The separation may inhibit fouling of the abrasive member; e.g., by allowing skin removed by the application of the skin treatment device to fall through the openings in the abrasive member and/or the separation between the body and the abrasive member.

In some implementations, the skin treatment device may include a body with a first member and a second member. The first member may include a handle and/or flanges. The second member may include an additional retention member, such as hooks. The hooks may be disposed on at least a portion of the bottom surface of the second member of the body. The abrasive member may include a mesh abrasive member. The mesh abrasive member may include a first surface that includes an abrasive; e.g., coupled to the mesh, and a second opposing surface. The second opposing surface may include a coupling member that removably couples with the additional retention member, such as loops. Thus, the loops of the mesh abrasive member may couple with the hooks of the second member of the body to couple the mesh abrasive member and the body. Thus, when a user decides to change an abrasive member; e.g., for a new abrasive member of the same or different type and/or for cleaning, the user may uncouple the loops of the mesh abrasive member from the hooks of the body to uncouple the abrasive member from the body. The user may then recouple the same or a different abrasive member by coupling the loops of the abrasive member with the hooks of the body; e.g., or vice versa with the loops of the body coupling with the hooks of the abrasive member.

In some implementations, the first member of the body may include flanges. The flanges may at least partially retain the abrasive member. In some implementations, the additional retention member; e.g., hooks, may provide the coupling of the abrasive member to the body without the use of the flanges. For example, the mesh abrasive member may couple to the body via a hook and loop coupling and the flanges may or may not be used to couple the mesh abrasive to the body. In some implementations, a first mesh abrasive member that is not coupled to the body via flanges may be removed; e.g., for replacement and/or cleaning, and a second abrasive member may then be coupled to the body via the flanges of the body and/or the additional retention member of the body; e.g., protrusions, hooks and/or loops.

The second member may include ridges. The ridges may increase the strength; e.g., rigidity, of the second member; e.g., when compared to a second member without ridges. In some implementations, the ridges may create a separation between a portion of the bottom surface of the second member and the abrasive member coupled to the body. The separation may allow skin, dirt, and/or debris that is removed from a part of the body, e.g., to which the skin treatment device is applied, to fall between openings in the abrasive member and/or through the separation.

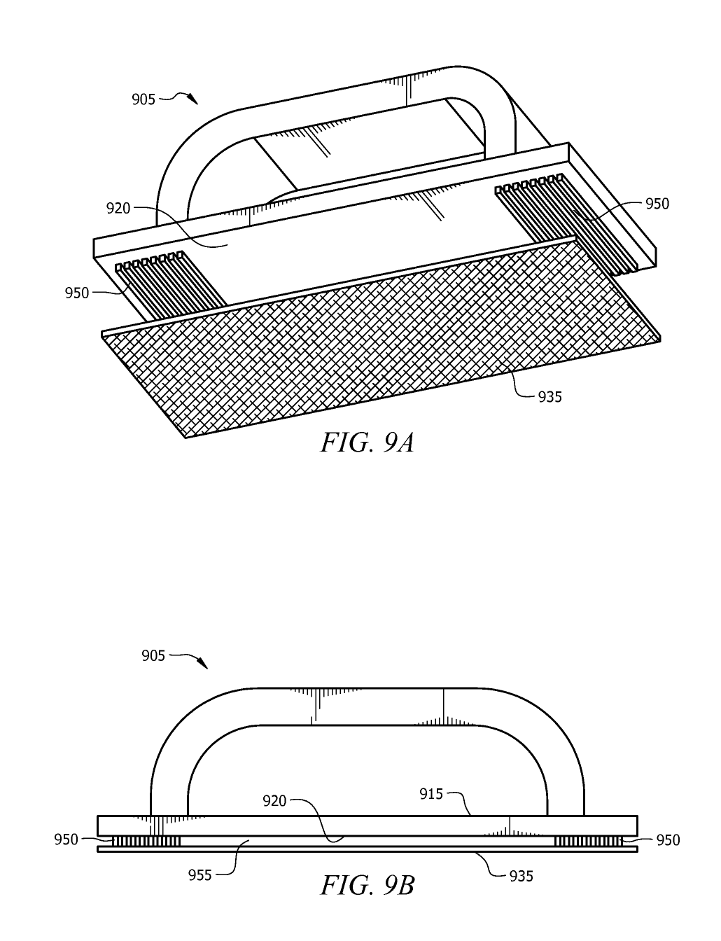

Referring to FIGS. 9A-9B, an implementation according to the present invention includes an abrasive sheet reversibly affixable to a bottom surface 920 of body 905.

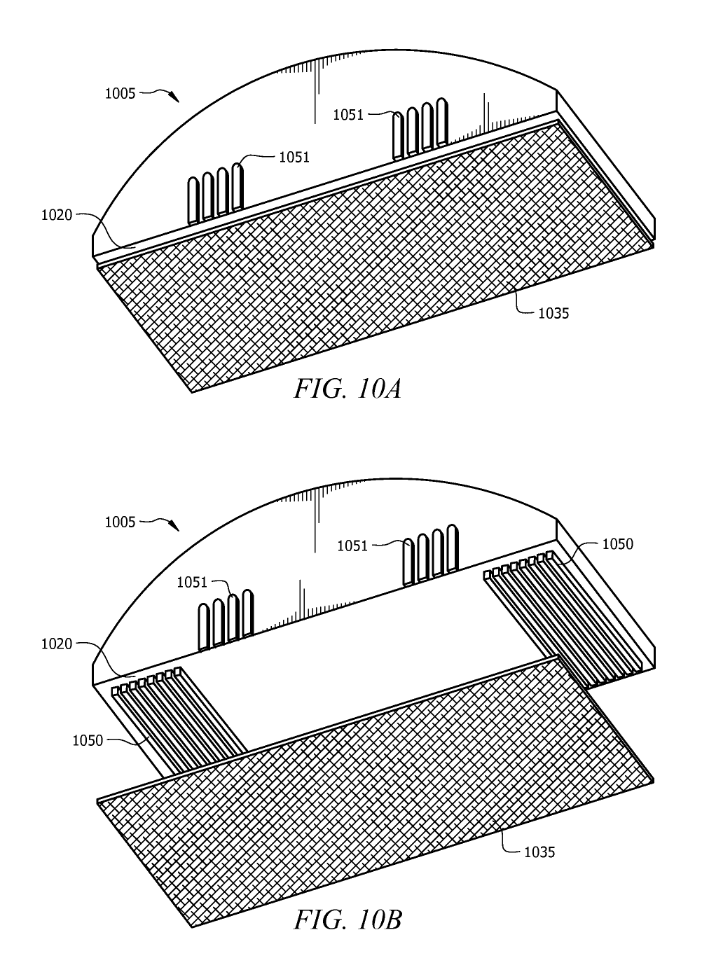

Bottom surface 920 may be rigid, semi-rigid or include deformable portions so that when pressed against a skin surface, the bottom surface may remain stiff or may deform slightly depending on the force applied by a user. The abrasive sheet is preferably affixable to a retaining member 950 that is raised or offset from surface 920 by a distance 955 that facilitates the movement of particulate matter through the abrasive sheet to an exit at sides of the device. Preferably, abrasive sheet 935 may contain perforations, apertures, gaps or may be of latticed or woven construction or a mesh to permit particulate matter to travel through the sheet. Abrasive sheet 935 may be coated with an adhesive on a side facing surface 920. Abrasive sheet 935 may be disposable or reusable. Retaining member 950 may be formed unitary with body 905, or added to surface 920 as a separate element. The retaining member may include series of projecting members in an arrayed or non-arrayed configuration wherein the projecting members may be flat, rounded or conical. The retaining member may include one or more raised ribs or ridges in rows. The retaining member may include a series of apertures or gaps within an otherwise solid structure. The retaining member may be coated with an adhesive which may be a light-tack type adhesive for temporary retention of the abrasive sheet, or the retaining member may include hooks or loops that affix to complimentary/mating structures on the back side of the abrasive sheet. Conversely, the abrasive sheet may be coated with an adhesive to temporarily adhere to the retaining member. In the implementation depicted in FIGS. 9A-9B, and FIGS. 10A-10B, the abrasive sheet has a substantially planar aspect when mounted and unstressed. Abrasive sheets may be supplied as pre-cut sheets of any suitable thickness for affixment to body 905, and may be supplied in various degrees of roughness. It is also conceivable that an abrasive sheet may have regions of differing roughness; e.g, different “grits.” It is conceivable that the abrasive sheets possess patterns (not shown) that correspond to regions of different roughness. While otherwise similar to the implementation depicted in FIGS. 9A-9B, the implementation depicted in FIGS. 10A-10B, includes at least a curved body 1005; wherein the body itself forms a handle portion. It will be appreciated by those having skill in the art and benefit of this disclosure that alternate handle types mounted to, or extending from a surface for the abrasive sheet to bear against, may be employed without departing from the invention. Also depicted in FIGS. 10A-B are grippers 1051. Grippers 1051 can comprise any device which aids the user in gripping the body 1005. Grippers 1051 can include raised protrusions which allow a user to grib the body 1005. The gripers 1051 can be straight, curved, rounded, circular, and comprise virtually any shape. Grippers 1051 help prevent the user's hand from undesirably sliding and losing control of the body 1005.

In some implementations, the skin treatment device may be lightweight. For example, ridges may be utilized rather than a solid body to provide rigidity for the use of the device. By utilizing the ridges to provide strength; e.g., rather than a solid second member, the weight of the skin treatment device may be decreased. The skin treatment device may include plastic and/or fabric members; e.g., rather than metal, and thus the weight of the skin treatment device may be decreased.

During use, the user may apply the skin treatment device to a portion of a body; e.g., user's body and/or another user's body, such that the mesh abrasive member contacts the portion of the body. The skin treatment device and/or the body may move such that the mesh abrasive member abrades at least a part of the portion of the body. For example, when a user applies the skin treatment device to a foot, the user may rub the bottom of the skin treatment device against the foot. Thus, at least a portion of the mesh abrasive member may contact and abrade the skin of the foot such that a part of the skin may be removed. The coupling between the loops; e.g., coupling member of the mesh abrasive member, and the hooks; e.g., additional retention member of the body, may have a strength to retain coupling of the abrasive member to the body during use. The skin may fall off the heel and/or fall through holes in the mesh abrasive member. Thus, fouling of the mesh abrasive member; e.g., coating of abrasive with skin, dirt, and/or debris during us may be inhibited by the openings in the mesh abrasive member. Cleaning the abrasive member may be facilitated by the openings, in some implementations. In one embodiment when skin is abraded, the abraded skin is held or retained by the abrasive member during operation. Thereafter, the device can be jostled or shaken to allow the abrasive member to release the abraded skin.

Although an end of the abrasive member has been described as being retained by the flange and/or disposed in a recess of the skin treatment device, other portions of the abrasive member may be retained by the flange and/or disposed in a recess. For example, a portion of the abrasive member may be folded an inserted into the recess. In some implementations, the abrasive member may include tabs or other coupling members that insert into the recess.

Although various features, such as surfaces and/or portions, have been described as top, bottom, and/or side, other orientations may be utilized in some implementations of the skin treatment devices. For example, the skin treatment device may be held during use such that the top surface is oriented downwards.

Although users have been described as a human, a user may be a person and/or a group of people. For example, a user may be a person that applies the skin treatment device to the skin of another person. A user may be a person to which the skin treatment is applied.

It is to be understood the implementations are not limited to particular systems or processes described which may, of course, vary. It is also to be understood that the terminology used herein is for the purpose of describing particular implementations only, and is not intended to be limiting. The singular terms “a”, “an”, and “the” include plural referents unless the context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting.

Although the present disclosure has been described in detail, it should be understood that various changes, substitutions and alterations may be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### Additional Description

The following clauses are offered as further description of the disclosed invention.
Clause 1. A skin treatment device comprising:
   a body comprising a first member coupled to a second member;
   wherein first member comprises at least one flexible flange;
   wherein said flexible flange is separated from said second member by a recess;
   wherein said flange comprises an initial position proximate a top surface of said second member and a retracted position wherein said flange is pulled upward from the top surface of said second member;
   an abrasive member at least partially disposed in said recess.
Clause 2. The skin treatment device of any proceeding or preceding clause wherein said abrasive member comprises a perforated abrasive member.
Clause 3. The skin treatment device of any proceeding or preceding clause wherein said first and second members comprise a single unit.
Clause 4. The skin treatment device of any proceeding or preceding clause wherein said first and second members are releasably coupled.
Clause 5. The skin treatment device of any proceeding or preceding clause comprising two opposing flanges.
Clause 6. The skin treatment device of any proceeding or preceding clause wherein said second member comprises ridges on a bottom surface of said second member.
Clause 7. The skin treatment device of any proceeding or preceding clause wherein said abrasive member is coupled to create a separation distance between a portion of a bottom surface of said second member and said abrasive member.
Clause 8. The skin treatment device of any proceeding or preceding clause wherein said body further comprises protrusions.
Clause 9. The skin treatment device of any proceeding or preceding clause wherein said protrusions comprise hooks, wherein said abrasive member comprises loops, and wherein said hooks couple with said loops.
Clause 10. The skin treatment device of any proceeding or preceding clause wherein said abrasive member comprises retaining members.
Clause 11. The skin treatment device of any proceeding or preceding clause wherein said second member comprises retaining members.
Clause 12. The skin treatment device of any proceeding or preceding clause wherein said abrasive member comprises a mesh abrasive member.
Clause 13. A skin treatment device comprising:
   a body comprising a bottom surface;
   a retaining member coupled to said bottom surface such that said retaining member projects downward from said bottom surface;
   an abrasive member removeably coupled to said retaining member, wherein said abrasive member is so coupled as to result in a separation between a portion of the bottom surface and the abrasive member.
Clause 14. The skin treatment device of any proceeding or preceding clause wherein said retaining member comprises a plurality of ridges located on the planar surface of the bottom surface.
Clause 15. The skin treatment device of any proceeding or preceding clause wherein said abrasive member comprises regions of differing roughness.
Clause 16. The skin treatment device of any proceeding or preceding clause wherein said body is curved.
Clause 17. The skin treatment device of any proceeding or preceding clause wherein said body comprises a handle.
Clause 18. The skin treatment device of any proceeding or preceding clause wherein said body comprises grippers.
Clause 19. The skin treatment device of any proceeding or preceding clause wherein said abrasive member comprises loops which hold abraded skin during operation and which releases said abraded skin upon jostling.

What is claimed is:

1. A skin treatment device comprising:
a body comprising a first member coupled to a second member;
wherein first member comprises at least one flexible flange;
wherein said flexible flange is separated from said second member by a recess;
wherein said flange comprises an initial position proximate a top surface of said second member and a retracted position wherein said flange is pulled upward from the top surface of said second member;
an abrasive member at least partially disposed in said recess.

2. The skin treatment device of claim 1 wherein said abrasive member comprises a perforated abrasive member.

3. The skin treatment device of claim 1 wherein said first and second members comprise a single unit.

4. The skin treatment device of claim 1 wherein said first and second members are releasably coupled.

5. The skin treatment device of claim 1 comprising two opposing flanges.

6. The skin treatment device of claim 1 wherein said second member comprises ridges on a bottom surface of said second member.

7. The skin treatment device of claim 1 wherein said abrasive member is coupled to create a separation distance between a portion of a bottom surface of said second member and said abrasive member.

8. The skin treatment device of claim 1 wherein said body further comprises protrusions.

9. The skin treatment device of claim 8 wherein said protrusions comprise hooks, wherein said abrasive member comprises loops, and wherein said hooks couple with said loops.

10. The skin treatment device of claim 1 wherein said abrasive member comprises retaining members.

11. The skin treatment device of claim 1 wherein said second member comprises retaining members.

12. The skin treatment device of claim 1 wherein said abrasive member comprises a mesh abrasive member.

13. A skin treatment device comprising:
a body comprising a bottom surface;
a retaining member coupled to said bottom surface such that said retaining member projects downward from said bottom surface;
an abrasive member removeably coupled to said retaining member, wherein said abrasive member is so coupled as to result in a separation between a portion of the bottom surface and the abrasive member.

14. The skin treatment device of claim 13 wherein said retaining member comprises a plurality of ridges located on the planar surface of the bottom surface.

15. The skin treatment device of claim 13 wherein said abrasive member comprises regions of differing roughness.

16. The skin treatment device of claim 13 wherein said body is curved.

17. The skin treatment device of claim 13 wherein said body comprises a handle.

18. The skin treatment device of claim 13 wherein said body comprises grippers.

19. The skin treatment device of claim 1 wherein said abrasive member comprises loops which hold abraded skin during operation and which releases said abraded skin upon jostling.
